(19) United States
(12) Patent Application Publication
(10) Pub. No.: US 2006/0211725 A1
(43) Pub. Date: Sep. 21, 2006

(54) Use

(75) Inventors: Masakazu Kobayashi, Glenview, IL (US); Hongsi Jiang, Wilmette, IL (US); Fan Pan, Wilmette, IL (US); Laurie Erickson, Chicago, IL (US); Aaron Ebbs, Reisterstown, MD (US); Carmen Wynn, Chicago, IL (US)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Astellas Pharma Inc., Chuo-ku (JP)

(21) Appl. No.: 11/319,178

(22) Filed: Dec. 28, 2005

Related U.S. Application Data
(63) Continuation of application No. 10/414,150, filed on Apr. 16, 2003, now abandoned.
(60) Provisional application No. 60/372,419, filed on Apr. 16, 2002.

(51) Int. Cl. A61K 31/4745; A61K 31/277; A61K 31/42

(52) U.S. Cl. 514/291; 514/378; 514/521

(57) ABSTRACT

This invention relates to a new use of a compound of the following formula (I) or (II) for the manufacture of a medicament for preventing and/or treating chronic rejection in a transplanted organ or tissue.

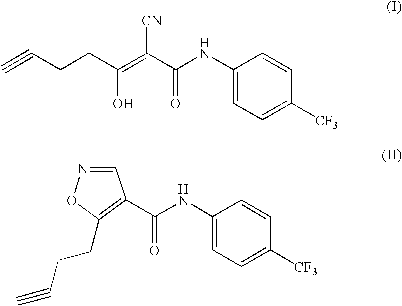

### TECHNICAL FIELD

This invention relates to a new use of a compound of the following formula (I) or (II) for the manufacture of a medicament for preventing and/or treating chronic rejection in a transplanted organ or tissue.

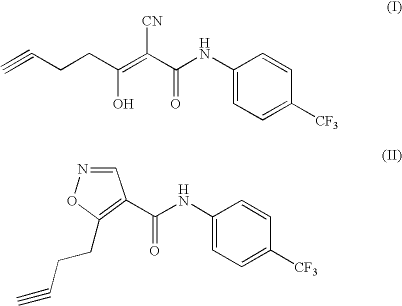

### BACKGROUND OF THE INVENTION

Organ transplants of liver, kidney, lung and heart are now regularly performed as treatment for endstage organ disease. Transplant outcome has progressively improved with the development of refinements in tissue typing, surgical techniques, and more effective immunosuppressive treatments. However, because of problems with chronic rejection, organ transplantation is not yet a clinically viable solution to irreversible organ disease.

Chronic rejection, which manifests as progressive and irreversible graft dysfunction, is one of the leading causes of late organ transplant loss in clinical transplantation.

The typical chronic rejection with the prognosis is an arteriosclerosis-like alteration, such as transplant vasculopathy, graft vessel disease, graft arteriosclerosis, transplant coronary disease, angiostenosis, interstitial fibrosis, etc. This vascular lesion is characterized by migration and proliferation of smooth muscle cells, namely, this leads to intimal proliferation and thickening, smooth muscle cell hypertrophy repair, and finally to gradual luminal obliteration (vascular remodelling). Especially, in the case of kidney, chronic rejection may be called chronic allograft nephropathy.

Chronic rejection appears to be inexorable and uncontrollable because there is no known effective treatment or prevention modality. Thus, there continues to exist a need for a remedy effective in preventing and/or treating chronic allograft rejection in clinical organ transplantation.

Concerning the compound (I) or (II) used in the present invention, it is known that the compound (I) or (II) is useful for the treatment of rheumatoid arthritis, chronic inflammatory diseases of immune or non-immune origin, and cancer in U.S. Pat. No. 5,308,865. While chronic inflammatory disease is disclosed in this patent, it is different from chronic rejection in a transplanted organ characterized by vascular lesion, so chronic rejection in a transplanted organ is not disclosed.

It is known that leflunomide and related compounds reduce overproliferation of smooth muscle cell following vascular injury, accordingly these compounds are useful for prevention and treatment of angiostenosis and arteriosclerosis following vascular injury in EP 0665013. However, the compound (I) or (II) of the present invention is not disclosed in the patent application. Additionally, chronic rejection in the present invention is discovered in whole vessel of transplanted organ as a result of host immune and non-immune responses, while the disease described in the patent application appears in injured part for damage restoration. So, these diseases are completely different on embryology in each other.

It is known that general leflunomide compounds have activities to control or reverse chronic rejection in a transplanted organ in U.S. Pat. No. 5,624,946 and U.S. Pat. No. 5,688,824. However, the compound (I) or (II) of the present invention is not disclosed in these patents.

Accordingly, it is not known at all that the compound (I) or (II) has activity to prevent and/or treat chronic rejection in a transplanted organ or tissue.

### DISCLOSURE OF THE INVENTION

The inventors of this invention have found that the compound (I) or (II) is effective for preventing and/or treating chronic rejection in a transplanted organ or tissue in a mammalian recipient.

Accordingly, this invention provides a new method for preventing and/or treating chronic rejection in a transplanted organ or tissue, which comprises administering a therapeutically effective amount of the compound (I) or (II) to a mammalian recipient in need thereof.

Further, this invention provides a new use of the compound (I) or (II) for the manufacture of a medicament for preventing and/or treating chronic rejection in a transplanted organ or tissue.

Still further, this invention provides anew pharmaceutical composition for preventing and/or treating chronic rejection in a transplanted organ or tissue, which comprises a therapeutically effective amount of the compound (I) or (II) in admixture with a pharmaceutically acceptable carrier or excipient.

A remedy capable of preventing chronic rejection is a remedy that prevents the occurrence of functional or histological signs of chronic rejection, when initiated before chronic rejection has commenced either by long term or short term administration. Therefore, preventing chronic rejection used in the present invention means protection or maintenance of transplanted organ or tissue for a long term.

The term “treatment” used in this invention means both treatments that comprise “controlling” and “reversing” the disease. And a treatment capable of controlling chronic rejection is a treatment that slows the progression of the disease process, when initiated after functional or histological signs of chronic rejection, respectively, are observed. Further, a treatment capable of reversing chronic rejection is a treatment that, when initiated after functional or histological signs of chronic rejection (respectively) have appeared, reverses the disease process and returns functional and histological findings closer to normal.

With respect to the compound (I), i.e. (2Z)-2-cyano-3-hydroxy-N-[4-(trifluoromethyl)phenyl]-2-hepten-6-ynamide, or the compound (II), i.e. 5-(3-butynyl)-N-[4-(trifluoromethyl) phenyl]-4-isoxazolecarboxamide, of the present invention, it can be produced according to the description in U.S. Pat. No. 5,308,865, Example 14 or a similar manner thereof, and it is to be understood that there may be a conformer and a stereoisomer, and such conformer and isomer are also included within the scope of this invention, and the compound (I) can be in another tautomer form. For example, the compound (I) can be either in its enol (I) or keto form (III), i.e. 2-cyano-3-oxo-N-[4-(trifluoromethyl)phenyl]-6-heptynami de, as shown in the following Scheme, and such a tautomer form is also included within the scope of this invention.

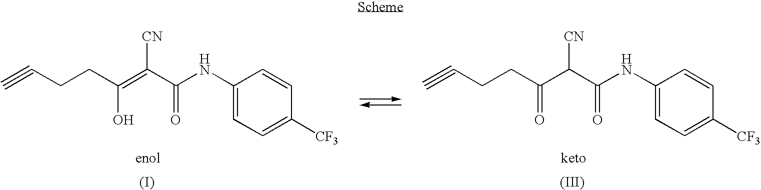

The compound (I) or (II) can be in a solvate, which is included within the scope of the present invention. The solvate preferably includes a hydrate and an ethanolate.

The compound (I) or (II) in the present invention can be used in the form of a pharmaceutical preparation, for example, in solid, semisolid or liquid form, which contains the compound (I) or (II) as an active ingredient, in admixture with an organic or inorganic carrier or excipient suitable for oral, parenteral such as intravenous, intramascular, subcutaneous or intraarticular, external such as topical, enteral, intrarectal, transvaginal, inhalant, ophthalmic, nasal or hypoglossal administration. The active ingredient may be compounded, for example, with the usual non-toxic, pharmaceutically acceptable, carriers for tablets, pellets, capsules, eye drops, suppositories, solutions (saline, for example), emulsion, suspensions (olive oil, for example), ointment, aerosol sprays, cream, skin plasters, patches and any other form suitable for use. The carriers which can be used are water, glucose, lactose, gum acacia, gelatin, mannitol, starch paste, magnesium trisilicate, corn starch, keratin, colloidal silica, potato starch, urea and other carriers suitable for use in manufacturing preparations, in solid, semisolid, or liquid form, and in addition auxiliary, stabilizing, thickening and coloring agents and perfumes may be used. The active object compound is included in the pharmaceutical composition in an effective amount sufficient to prevent and/or treat chronic rejection in a transplanted organ or tissue.

Mammals which may be treated in the present invention include livestock mammals such as cows, houses, etc., domestic animals such as dogs, cats, rats, etc. and humans, preferably humans.

Organs or tissues may be transplanted from a donor to a recipient of same individual (autograft), syngeneic species (isograft), the same species (allograft) or different species (xenograft). Such transplanted organs or tissues may be liver, kidney, heart, lung, combined heart-lung, trachea, spleen, pancreatic (complete or partial, e.g. Langerhans islets), skin, small intestine, cornea, bone marrow, limb, muscle, nerve, intervertebral disc, myoblast or cartilage, or a combination of any of the foregoing.

The compound (I) or (II) for use in the preventing and/or treating of chronic rejection may be administered alone or in combination with one or more other immunosuppressive agents, for example cyclosporin A, tacrolimus, rapamycin, azathioprine, corticosteroids, anti-lymphocyte globulin or OKT3; especially cyclosporin A or tacrolimus, simultaneously, separately or sequentially. Further, the compound (I) or (II) for this use can be administered in a form of mixture in a pharmaceutical composition with one or more other immunosuppressive agents, mentioned above. Such combination or mixing remedy is included within the scope of this invention.

While the dosage of therapeutically effective amount of the compound (I) or (II) varies from and also depends upon the age and condition of each individual patient to be treated, a daily dose of about 1 mg-10 g/body, preferably 5 mg-5 g/body and more preferably 10 mg-2 g/body of the active ingredient is generally given for preventing and/or treating this disease, and an average single dose of about 0.5-1 mg, 5 mg, 10 mg, 50 mg, 100 mg, 250 mg, 500 mg, 1 g, 2 g and 3 g is generally administered. Daily dose for administration in humans for preventing or treating chronic rejection will be in the range of about 0.1-50 mg/kg. In a combination or mixing remedy, for example, tacrolimus may be administered in humans in a daily dose of about 0.01-5 mg/kg, preferably 0.05-0.5 mg/kg.

While the term for administering the compound (I) or (II) to prevent chronic rejection varies depending on species, and the nature and severity of the condition to be prevented, the compound (I) or (II) may usually be administered to humans for a short term or a long term, i.e. for 1 week to 1 year or more after transplantation, unless chronic rejection commences.

The possible mechanism of preventing and treating of chronic rejection in the compound (I) or (II) is associated with reduction of anti-glomeruli basement membrane (GBM) antibody, following by a sustained suppression of TGFβ.

The following examples illustrate the present invention in further detail. It should be understood that those examples are not intended to limit the scope of the invention.

### EXAMPLE 1

### Prevention of Chronic Rejection

(1) Method

Inbred male Lewis rats (LEW) (RT1^{I}), weighing 250-300 g, were used as kidney transplantation recipients. Inbred male LEW and Fisher (F344) (RT1^{IvI}), weighing 250-350 g, were used as isograft and allograft donor rats, respectively. Kidney transplantation was performed using the modified technique of Fisher and Lee. [Fisher et al., Surgery, 58:904-914, 1965] Survival of kidney transplant was measured as time of recipient rat survival. Blood and 24 hr urine samples were collected once a week for plasma creatinine, proteinuria, and the measurement of antibody titer against donor glomeruli basement membrane protein (GBM). Kidney grafts were harvested on the 90^{th} day posttranspalantation and subjected to histology and reverse transcriptase-polymerase chain reaction (RT-PCR) analysis. The compound (I), at doses of 10 mg/kg and 20 mg/kg were administered orally to recipient rats daily from day 0 to day 9 after transplantation. Control isograft and allograft recipients received no drug after transplantation.

The recipient's kidney function was determined by measuring their plasma creatinine and proteinuria once a week for 90 days. Blood and urine samples were collected from recipients with kidney grafts described in the above. Plasma creatinine was tested by Sigma Creatinine Kit and proteinuria by Bio-Rad Protein assay.

Kidney graft tissues were harvested from recipients on day 90th after transplantation for histological analysis. Graft samples were fixed in 10% NBF and subsequently processed then immediately embedded in ParaPlast™ paraffin embedding media. Samples were sectioned at 3 μm, pre-warmed, deparaffinized, rehydrated, and subsequently stained in one of four processes: Hematoxylin and Eosin, Per-Iodic Acid Schiff, Verhoeff's Combined Elastic Trichrome, and Per-Iodic Acid Silver Methenamine. Histological sections were blindly evaluated by two histologists and scored semiquantitatively based on modified Banff' criteria for transplant pathology. [Solez et al., Kidney Int., 44:411-422, 1993]

TGFβ has been considered to play a crucial role for causing chronic allograft rejection. Kidney graft tissues harvested from recipients on day 90th after transplantation were subjected to RT-PCR for TGFβ gene expression. Total RNA was extracted from transplanted kidney tissues by TRIZOL. Real time RT-PCR was performed as described by Overbergh et al, [Overbergh et al., Cytokine, 11:305, 1999] using the ABI Prism 7700 sequence detection system and reagents from PE Biosystems, normalized to rodent GAPDH. The primers and probe for rat TGFβ were 5′-GCTGCTGACCCCCACTGAT-(sense), 5′-GCCACTGCCGGACAACTC-(anti sense), and CGCCTGAGTGGCTGTCTTTTGACGT-TAMRA. Rodent GAPDH primers and probe were designed by PE Biosystem.

Specific antibody against F344 rat glomeruli basement membrane protein in plasma from LEW recipients with F344 kidneys were also measured in the isograft, untreated allograft and allograft treated with the compound (I) at doses of 10 mg/kg and 20 mg/kg near days 20, 40, and 90 after transplantation by using ELISA assay.

(2) Result

The isografts survived more than 90 days. In contrast, only 40% of the control allografts survived more than 90 days after grafting. The allografts of those receiving the compound (I) at dose of 10 mg/kg and the compound (I) at dose of 20 mg/kg survived more than 90 days post-transplantation were 80% and 100%, respectively. (Table 1)

TABLE 1

| | | | | | Survival | Survival |
|---|---|---|---|---|---|---|
| Group | Drug | Route | Period | n | day | rate |
| Isograft | — | — | — | 6 | >90 | 100% |
| Allograft | — | — | — | 10 | 14, 20, 21, | 40% |
| | | | | | 24, 38, | |
| | | | | | 72, >90(4) | |
| Compound (I) | 10 mg/kg | PO | 0-9 day | 5 | 28, >90(4) | 80% |
| Compound (I) | 20 mg/kg | PO | 0-9 day | 5 | >90(5) | 100% |

In the absence of the compound (I) treatment, recipient plasma creatinine was increased by week 7 and proteinuria was positively detected by week 5. Both the compound (I) at doses of 10 mg/kg and 20 mg/kg treated recipients maintained normal creatinine and undetectable proteinuria as in the naive rats and the isograft recipients during the period we followed. (FIG. 1-4)

The untreated allograft control was observed for development of progressive histological chronic rejection. The approximate cumulative reduction in Banff' scores of kidney grafts from recipients treated with the compound (I) 10 mg/kg and 20 mg/kg are as following: interstitial inflammation 50% and 67%, tubulitis 100% and 100%, vasculitis 33% and 50%, mesangiolysis 83% and 100%, glomerulitis 75% and 38%, tubular atrophy 40% and 85%, glomerulosclerosis 83% and 100%, fibro-intimal hyperplasia 63% and 44%, and transplant glomerulopathy 79% and 100%, respectively, when compared with the untreated allograft control. And based on Banff' criteria of kidney transplant pathology, (−):Grade 0, Normal, (+):Grade 1, Mild, (++):Grade 2, Moderate and (+++):Grade 3, Severe are used for diagnostic evaluation of chronic rejection. (Table 2)

TABLE 2

| Group | 1* | 2* | 3* | 4* | 5* | 6* | 7* | 8* | 9* |
|---|---|---|---|---|---|---|---|---|---|
| Compound | + | − | + | − | + | + | − | ++ | − |
| (I) | | | | | | | | | |
| 10 mg from | | | | | | | | | |
| day 0-9 | | | | | | | | | |
| Compound | ++ | − | +++ | + | − | + | + | − | + |
| (I) | | | | | | | | | |
| 10 mg from | | | | | | | | | |
| day 0-9 | | | | | | | | | |
| Compound | + | − | +++ | − | ++ | + | − | ++ | − |
| (I) | | | | | | | | | |
| 20 mg from | | | | | | | | | |
| day 0-9 | | | | | | | | | |
| Compound | + | − | +++ | − | + | − | − | ++ | − |
| (I) | | | | | | | | | |
| 20 mg from | | | | | | | | | |
| day 0-9 | | | | | | | | | |
| Compound | + | − | − | − | − | − | − | + | − |
| (I) | | | | | | | | | |
| 20 mg from | | | | | | | | | |
| day 0-9 | | | | | | | | | |
| Compound | + | − | − | − | ++ | − | − | + | − |
| (I) | | | | | | | | | |
| 20 mg from | | | | | | | | | |
| day 0-9 | | | | | | | | | |
| Allograft | +++ | + | +++ | +++ | + | + | +++ | ++ | ++ |
| Control | | | | | | | | | |
| Allograft | +++ | ++ | +++ | +++ | +++ | ++ | +++ | +++ | ++ |
| Control | | | | | | | | | |
| Allograft | +++ | ++ | +++ | +++ | ++ | ++ | +++ | +++ | +++ |
| control | | | | | | | | | |

1*: Inflammation,
2*: Tubulitis,
3*: Vasculitis,
4*: Mesangiolysis,
5*: Glomerulitis,
6*: Tubular Atrophy,
7*: Glomerulosclerosis,
8*: Fibro-intimal Hyperplasia,
9*: Transplant Glomerulopathy

Compared with the isograft control, TGFβ mRNA was significantly up-regulated in the untreated allograft control. The compound (I) treatment inhibited TGFβ gene expression in a dose-dependent manner on day 90 after grafting compared with the untreated allograft control. (FIG. 5)

In the isograft control group, plasma anti-GBM was undetectable. It was detectable near day 20 after transplantation, increased thereafter in the untreated allograft control. Both the compound (I) at doses of 10 mg/kg and 20 mg/kg—treated recipients showed a trend of reduced production of antibody against donor GBM. (FIG. 6-9)

### EXAMPLE 2

### Prevention of Chronic Rejection in Combination with Tacrolimus

(1) Method

The rats and kidney transplantation methods described in Example 1 were used. The compound (I) at dose of 3 mg/kg and tacrolimus at dose of 1 mg/kg, were administered orally to recipient rats daily for 90 days after transplantation. The isograft, untreated allograft, and allograft treated with tacrolimus 1 mg/kg for 90 days alone served as control groups.

Blood and urine samples were collected once a week for 90 days from recipients with kidney grafts described in Example 1 for measuring their plasma creatinine and proteinuria. Plasma creatinine was tested by Sigma Creatinine Kit and proteinuria by Bio-Rad Protein assay.

Using the methods described in Example 1, histological changes of chronic allograft rejection were analyzed. Histological sections were blindly evaluated by two histologists and scored semiquantitatively based on modified Banff' criteria for transplant pathology.

Specific antibody against F344 rat glomeruli basement membrane protein in plasma from LEW recipients with F344 kidneys were also measured in the isograft, untreated allograft and allograft treated with the compound (I) at dose of 3 mg/kg, in combination with tacrolimus at dose of 1 mg/kg near day 20, 40, and 90 after transplantation by using methods described in Example 1.

(2) Result

The isografts survived more than 90 days. In contrast, only 40% of the control allografts survived 90 days after grafting. The allografts of those receiving tacrolimus at dose of 1 mg/kg and the compound (I) at dose of 3 mg/kg in combination with tacrolimus at dose of 1 mg/kg survived 90 days posttransplantation were both 100%. (Table 3)

TABLE 3

| | | | | | Survival | Survival |
|---|---|---|---|---|---|---|
| Group | Drug | Route | Period | n | day | rate |
| Isograft | — | — | — | 6 | >90 | 100% |
| Allograft | — | — | — | 10 | 14, 20, 21, | 40% |
| | | | | | 24, 38, | |
| | | | | | 72, >90(4) | |
| Tacrolimus | 1 mg/kg | PO | 0-90 day | 4 | >90 | 100% |
| Compound (I) | 3 mg/kg | PO | 0-90 day | 4 | >90 | 100% |
| Tacrolimus | 1 mg/kg | PO | | | | |

In the untreated allogenic transplantation, recipient plasma creatinine was increased by week 7 and proteinuria was positively detected by week 5. The compound (I) at dose of 3 mg/kg in combination with tacrolimus at dose of 1 mg/kg—treated recipients showed decreased levels in both plasma creatinine and proteinuria compared with the untreated allograft control. (FIGS. 10, 11)

The untreated allograft control was observed for development of progressive histological chronic rejection. The approximate cumulative reduction in Banff' scores of kidney grafts from recipients treated with the compound (I) at dose of 3 mg/kg and tacrolimus at dose of 1 mg/kg are as following: interstitial inflammation 50%, tubulitis 85%, vasculitis 92%, mesangiolysis 75%, glomerulitis 38%, tubular atrophy 55%, glomerulosclerosis 58%, fibro-intimal hyperplasia 63%, and transplant glomerulopathy 57%, respectively, when compared with the untreated allograft control. And based on Banff' criteria of kidney transplant pathology, (−), (+), (++) and (+++) are defined same as Table 2. (Table 4)

TABLE 4

| Group | 1* | 2* | 3* | 4* | 5* | 6* | 7* | 8* | 9* |
|---|---|---|---|---|---|---|---|---|---|
| Compound (I) 3 mg + Tacrolimus | + | − | + | − | + | + | − | ++ | − |
| 1 mg for 90 days | | | | | | | | | |
| Compound (I) 3 mg + Tacrolimus | ++ | − | +++ | + | − | + | + | − | + |
| 1 mg for 90 days | | | | | | | | | |
| Compound (I) 3 mg + Tacrolimus | + | − | +++ | − | ++ | + | − | ++ | − |
| 1 mg for 90 days | | | | | | | | | |
| Compound (I) 3 mg + Tacrolimus | + | − | +++ | − | + | − | − | ++ | − |
| 1 mg for 90 days | | | | | | | | | |
| Allograft Control | +++ | + | +++ | +++ | + | + | +++ | ++ | ++ |
| Allograft Control | +++ | ++ | +++ | +++ | +++ | ++ | +++ | +++ | ++ |
| Allograft control | +++ | ++ | +++ | +++ | ++ | ++ | +++ | +++ | +++ |

1*: Inflammation,
2*: Tubulitis,
3*: Vasculitis,
4*: Mesangiolysis,
5*: Glomerulitis,
6*: Tubular Atrophy,
7*: Glomerulosclerosis,
8*: Fibro-intimal Hyperplasia,
9*: Transplant Glomerulopathy

In the isograft control group, plasma anti-GBM was undetectable. It was detectable by day 20 after transplantation, increase thereafter in the untreated allograft control. The compound (I) at dose of 3 mg/kg, in combination with tacrolimus at dose of 1 mg/kg—treated recipients had no detectable levels of antibody against donor GBM, as in the isograft control group. (FIG. 12)

### EXAMPLE 3

### Treatment of Chronic Rejection

(1) Method

The rats and kidney transplantation methods described in Example 1 were used. The compound (I) at a dose of 20 mg/kg was administered orally to recipient rats for 3 weeks started from the time when they revealed either increased plasma creatinine or detectable proteinuria. The isograft and untreated allograft served as control groups. Blood and urine samples were collected once a week from recipients with kidney grafts described in Example 1 for measuring their plasma creatinine and proteinuria. Plasma creatinine was tested by Sigma Creatinine Kit and proteinuria by Bio-Rad Protein assay.

Using the methods described in Example 1, histological changes of chronic allograft rejection under rescue treatment of the compound (I) were analyzed. Histological sections were blindly evaluated by two histologists and scored semiquantitatively based on modified Banff' criteria for transplant pathology.

(2) Result

In the untreated allograft control, recipient plasma creatinine was increased by week 7 and proteinuria was positively detected by week 5. Although the compound (I) rescue treatment did not show an immediate improvement of recipient kidney function, both plasma creatinine and proteinuria tended to be at a normal level after drug treatment was discontinued. (FIGS. 13, 14)

The untreated allograft control was observed for development of progressive histological chronic rejection. The approximate cumulative reduction in Banff' scores of kidney grafts from recipients treated with the compound (I) at dose of 20 mg/kg for 3 weeks during ongoing chronic allograft rejection are as following: interstitial inflammation 50%, tubulitis 70%, vasculitis 92%, mesangiolysis 33%, glomerulitis 38%, tubular atrophy 42%, fibro-intimal hyperplasia 53%, and transplant glomerulopathy 89%, respectively, when compared with the untreated allograft control. And based on Banff' criteria of kidney transplant pathology, (−), (+), (++) and (+++) are defined same as Table 2. (Table 5)

TABLE 5

| Group | 1* | 2* | 3* | 4* | 5* | 6* | 7* | 8* | 9* |
|---|---|---|---|---|---|---|---|---|---|
| Compound (I) rescue | + | − | + | − | + | + | − | ++ | − |
| From day 40-70 | | | | | | | | | |
| Compound (I) rescue | ++ | − | +++ | + | − | + | + | − | + |
| From day 40-70 | | | | | | | | | |
| Compound (I) rescue | + | − | +++ | − | ++ | + | − | ++ | − |
| From day 40-70 | | | | | | | | | |
| Compound (I) rescue | + | − | +++ | − | + | − | − | ++ | − |
| From day 40-70 | | | | | | | | | |

1*: Inflammation,
2*: Tubulitis,
3*: Vasculitis,
4*: Mesangiolysis,
5*: Glomerulitis,
6*: Tubular Atrophy,
7*: Glomerulosclerosis,
8*: Fibro-intimal Hyperplasia,
9*: Transplant Glomerulopathy

### EXAMPLE 4

### Treatment of Chronic Rejection in Combination with Brief Treatment of Tacrolimus

(1) Method

The rats and kidney transplantation methods described in Example 1 were used. Tacrolimus at dose of 1 mg/kg from day 0 to day 9 after transplantation, and the compound (I) at doses of 10 mg/kg and 15 mg/kg from day 28 today 60 after transplantation were administered orally to recipient rats. In this study LEW recipients were briefly treated with oral tacrolimus at 1 mg/kg/day for 10 days after transplantation to avoid acute rejection and slow chronic rejection that gradually destroys the F344 kidney graft, resulting in functional and histological changes similar to the chronic rejection in human. The isograft, untreated allograft and allograft treated with tacrolimus 1 mg/kg for 10 days alone served as control groups. Blood and urine samples were collected once a week from recipients with kidney grafts described in Example 1 for measuring their plasma creatinine and proteinuria. Plasma creatinine was tested by Sigma Creatinine Kit and proteinuria by Bio-Rad Protein assay.

(2) Result

The isografts survived more than 90 days. In contrast, only 40% of the control allografts survived up to 90 days after grafting. The allografts of those receiving tacrolimus at dose of 1 mg/kg for 10 days alone after transplantation showed 100% of allograft survival rate. The individual allograft survival rates for recipients treated with a brief dose of tacrolimus and the compound (I) 10 mg/kg or 15 mg/kg from day 28 to day 60 after transplantation will be available after increasing of animal case number. (Table 6)

TABLE 6

| | | | | | Survival | |
|---|---|---|---|---|---|---|
| Group | Drug | Route | Period | n | day | Survival rate |
| Isograft | — | — | — | 6 | >90 | 100% |
| Allograft | — | — | — | 10 | 14, 20, 21, 24, 38, | 40% |
| | | | | | 72, >90(4) | |
| Tacrolimus | 1 mg/kg | PO | 0-9 day | 5 | >90 | 100% |
| Tacrolimus | 1 mg/kg | PO | 0-9 day | 2 | >90(2) | N/A |
| Compound (I) | 10 mg/kg | PO | 28-60 day | | | |
| Tacrolimus | 1 mg/kg | PO | 0-9 day | 1 | >90 | N/A |
| Compound (I) | 15 mg/kg | PO | 28-60 day | | | |

The recipient's kidney function was determined by measuring their plasma creatinine and proteinuria once a week for 90 days. Plasma creatinine increased rapidly after week 7 post transplantation in the allograft control and week 8 in the allografts treated with a brief dose of tacrolimus, whereas, is remained within the normal range in the isograft control. The compound (I) 10 mg/kg from day 28 to day 60 maintained the plasma creatinine level less than the normal value of 1.5 mg/dL during the entire study period. Although the recipient treated with the compound (I) 15 mg/kg/day showed increased plasma creatinine started from week 3 to week 9 after transplantation, it was reversed and maintained in a normal level after that. (FIGS. 15, 16) Among the 40% of the allograft control rats and 100% of the allografts treated with a brief dose of tacrolimus survived more than 90 days after transplantation, preteinuria were detectable by week 2 and week5, respectively after transplantation and dramatically increasing thereafter when compared with the isograft control. Both the compound (I) 10 mg/kg and 15 mg/kg treatment from day 28 to day 60 decreased the progression of proteinuria in kidney recipients. (FIGS. 17, 18)

The compound (I) or (II) was proved to have an activity to prevent and/or treat chronic rejection in a transplanted organ or tissue. So, the present invention provides useful immunosuppressant for preventing and/or treating chronic rejection in a transplanted organ or tissue.

### BRIEF DESCRIPTION OF THE DRAWINGS

FIG. 1 shows plasma creatinine concentrations after treatment with the compound (I) at dose of 10 mg/kg. (Example 1)

FIG. 2 shows plasma creatinine concentrations after treatment with the compound (I) at dose of 20 mg/kg. (Example 1)

FIG. 3 shows proteinuria quantities after treatment with the compound (I) at dose of 10 mg/kg. (Example 1)

FIG. 4 shows proteinuria quantities after treatment with the compound (I) at dose of 20 mg/kg. (Example 1)

FIG. 5 shows inhibition of TGFβ gene expression in treatment with the compound (I). (Example 1)

FIG. 6 shows productions of antibody against GBM in syngeneic transplantation. (Example 1)

FIG. 7 shows productions of antibody against GBM in allogeneic transplantation. (Example 1)

FIG. 8 shows productions of antibody against GBM in allogeneic transplantation treated with the compound (I) at dose of 10 mg/kg. (Example 1)

FIG. 9 shows productions of antibody against GBM in allogeneic transplantation treated with the compound (I) at dose of 20 mg/kg. (Example 1)

FIG. 10 shows plasma creatinine concentrations in transplantation treated with the compound (I) at dose of 3 mg/kg in combination with tacrolimus at dose of 1 mg/kg. (Example 2)

FIG. 11 shows proteinuria quantities in transplantation treated with the compound (I) at dose of 3 mg/kg in combination with tacrolimus at dose of 1 mg/kg. (Example 2)

FIG. 12 shows productions of antibody against GBM in allogeneic transplantation treated with the compound (I) in combination with tacrolimus. (Example 2)

FIG. 13 shows plasma creatinine concentrations in transplantation treated with rescue the compound (I) at dose of 20 mg/kg. (Example 3)

FIG. 14 shows proteinuria quantities in transplantation treated with rescue the compound (I) at dose of 20 mg/kg. (Example 3)

FIG. 15 shows plasma creatinine concentrations in transplantation treated with the compound (I) at dose of 10 mg/kg with brief treatment of tacrolimus. (Example 4)

FIG. 16 shows plasma creatinine concentrations in transplantation treated with the compound (I) at dose of 15 mg/kg with brief treatment of tacrolimus. (Example 4)

FIG. 17 shows proteinuria quantities in transplantation treated with the compound (I) at dose of 10 mg/kg with brief treatment of tacrolimus. (Example 4)

FIG. 18 shows proteinuria quantities in transplantation treated with the compound (I) at dose of 15 mg/kg with brief treatment of tacrolimus. (Example 4)

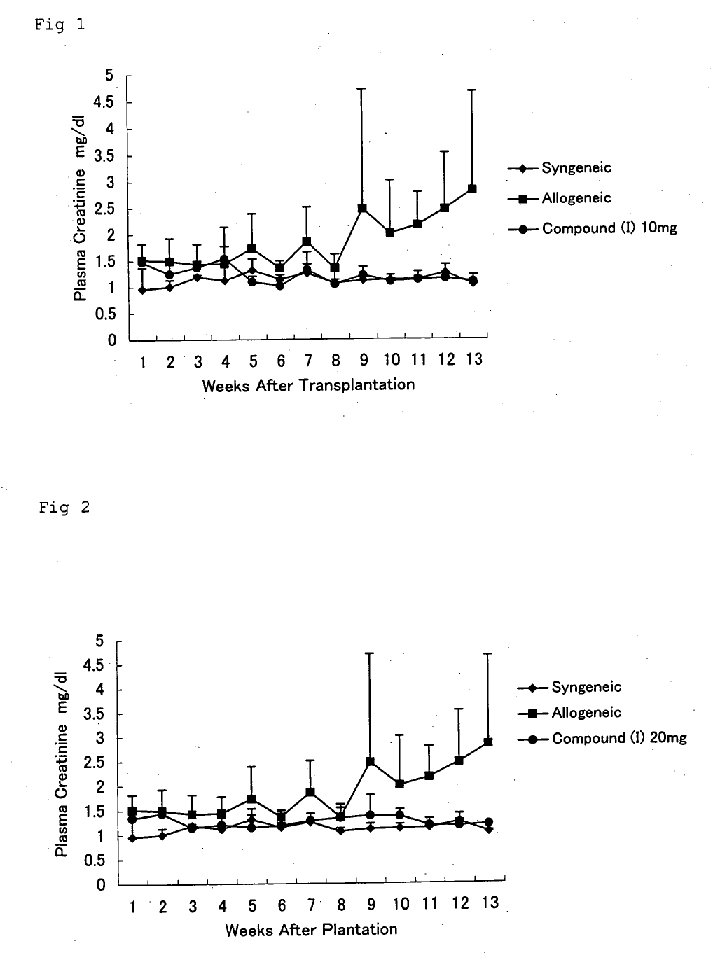

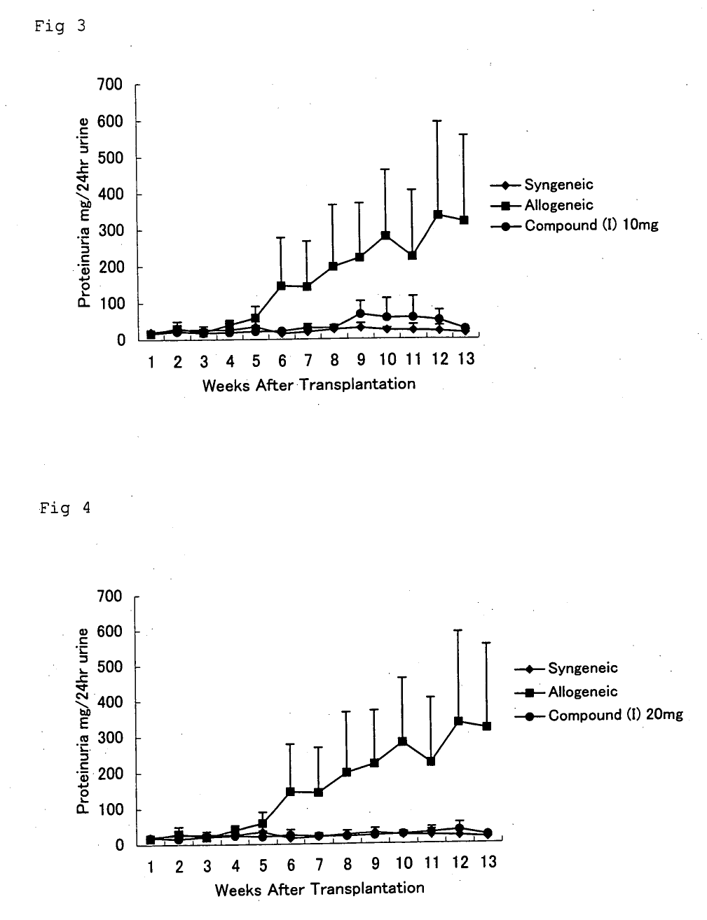

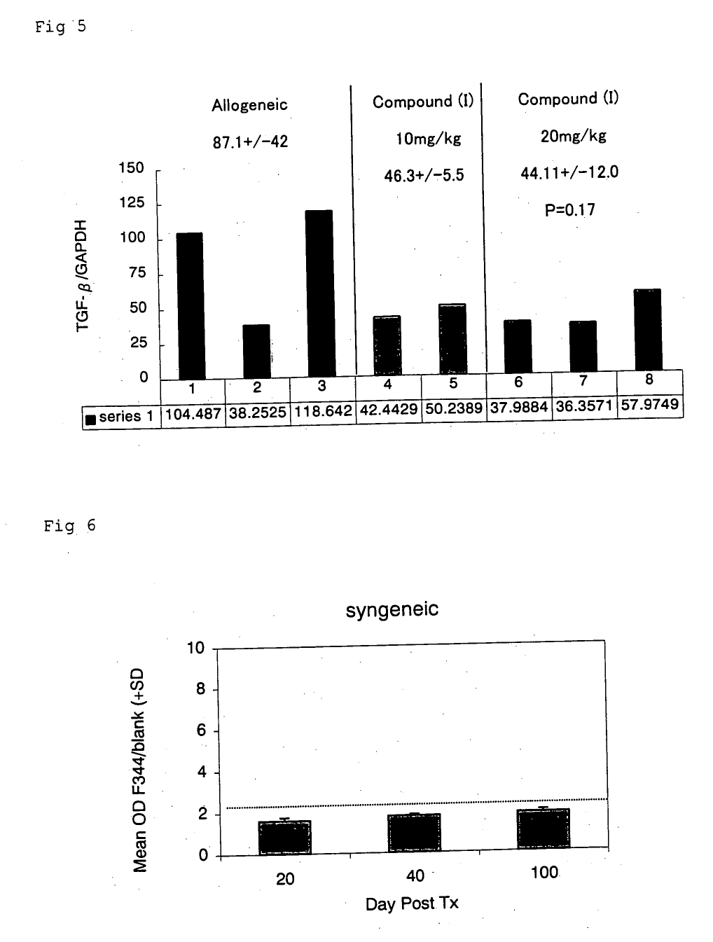

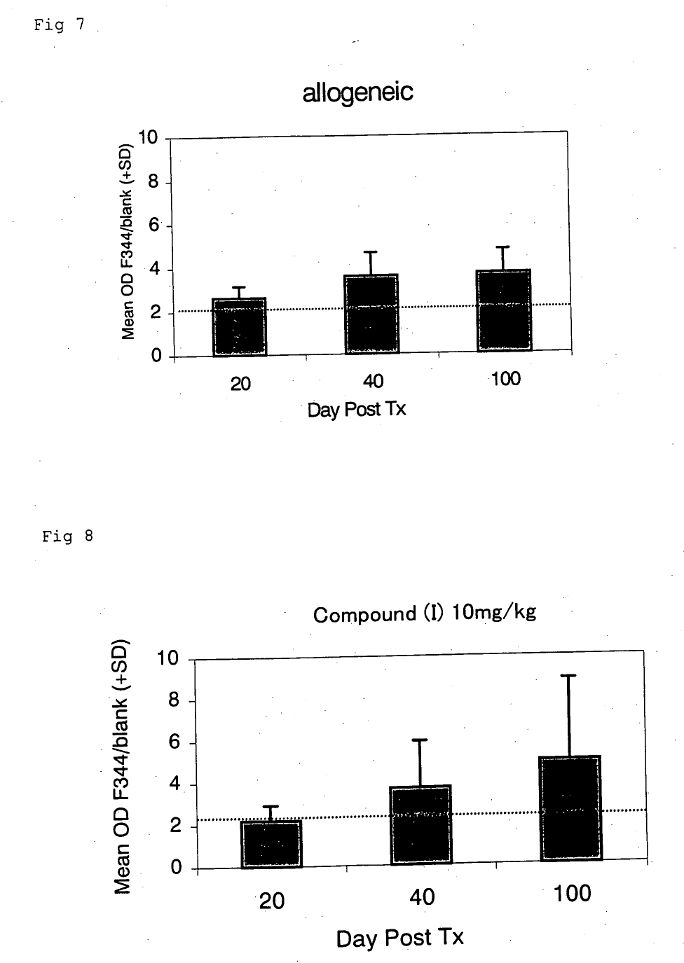

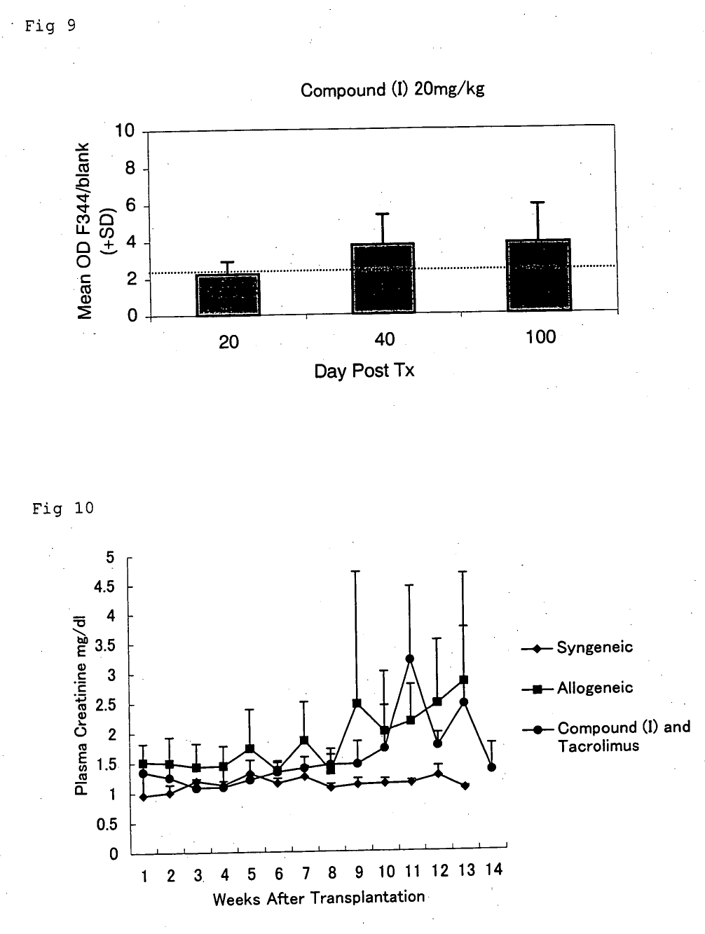

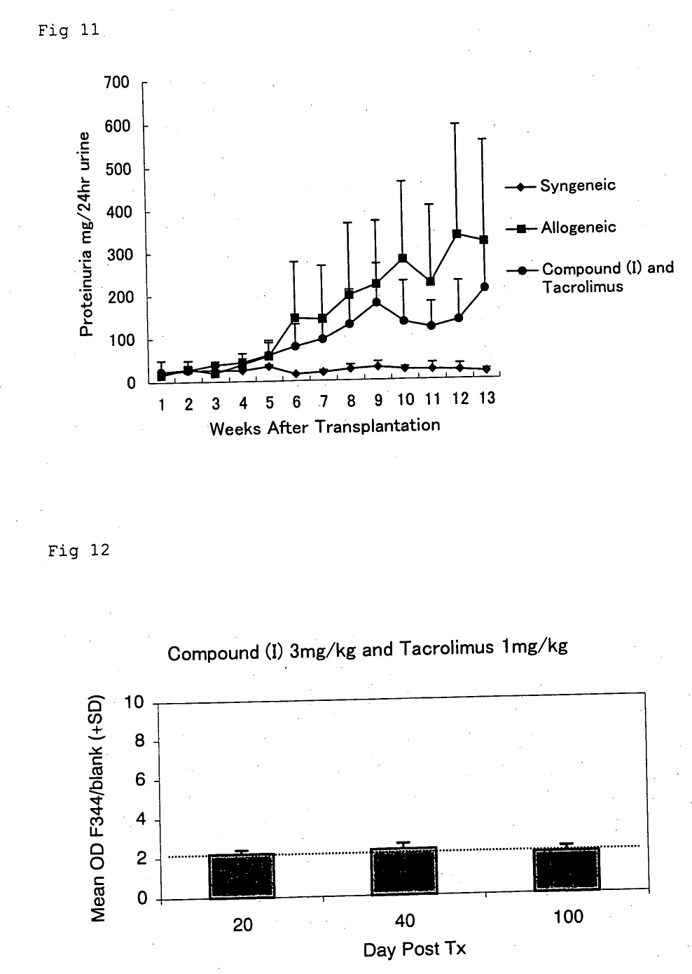

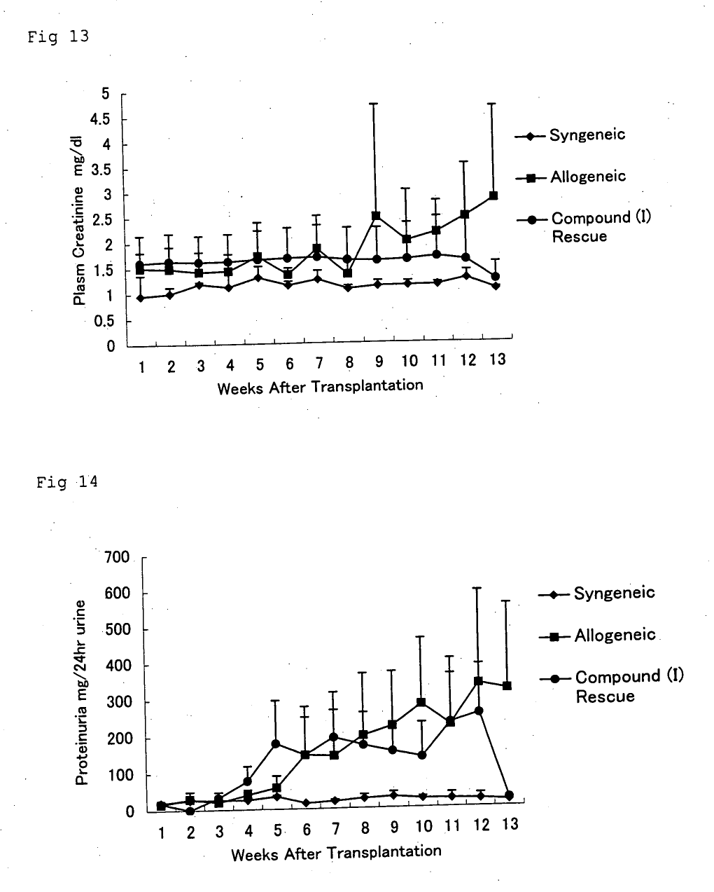

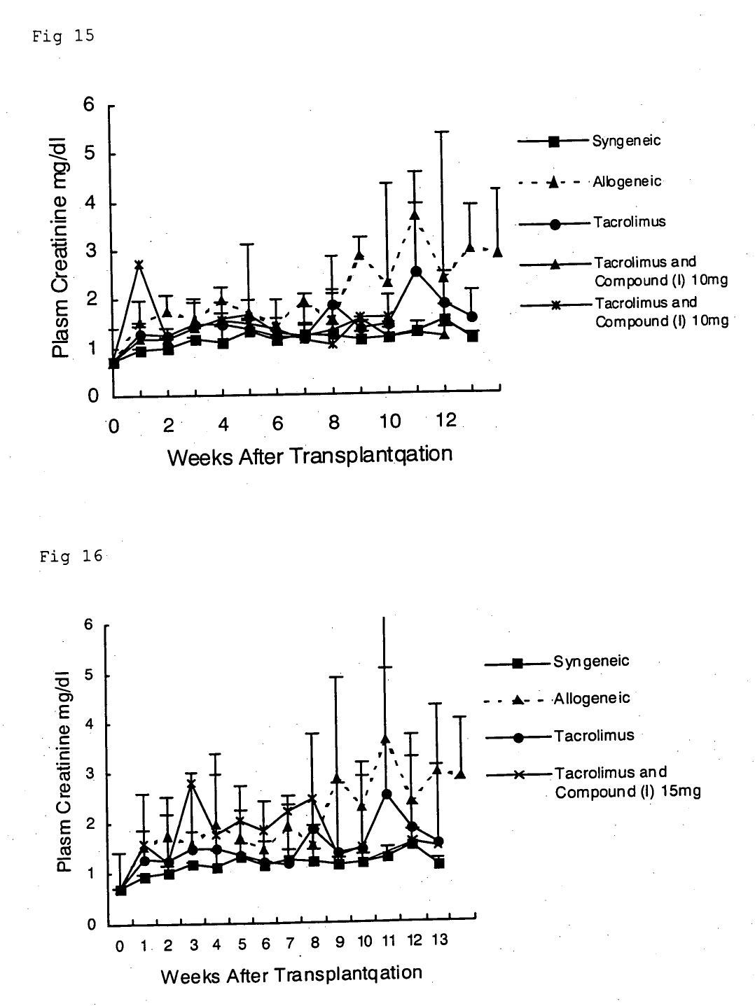

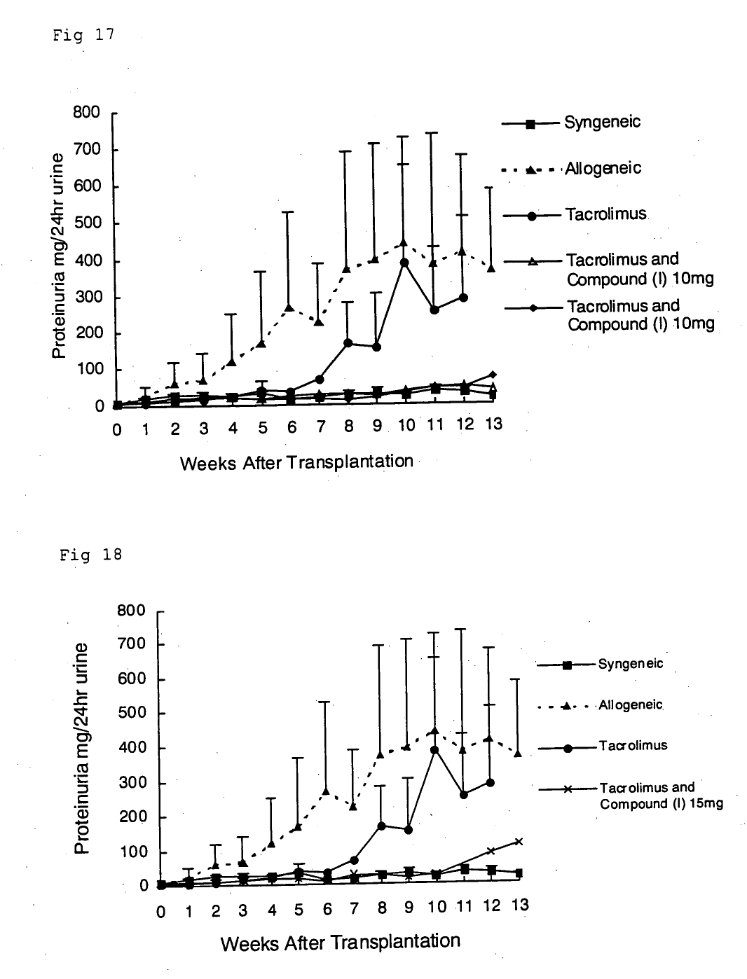

1. A method for preventing and/or treating chronic rejection in a transplanted organ or tissue, which comprises administering a therapeutically effective amount of compound of the formula (I) or (II): 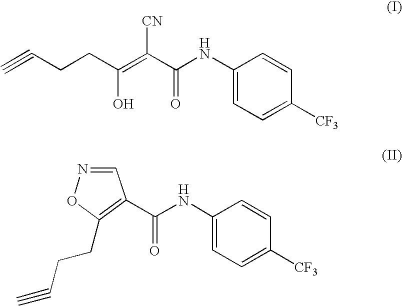
to a mammalian recipient in need thereof.

2. The method of claim 1 wherein the method is for preventing chronic rejection.

3. The method of claim 2 wherein the transplantation is allograft transplantation.

4. The method of claim 1 further comprising administering a therapeutically effective amount of tacrolimus.

5. The method of claim 3 further comprising administering a therapeutically effective amount of tacrolimus.

6. The method of claim 1 wherein the method is in oral administration.

7. A use of a compound of the formula (I) or (II): 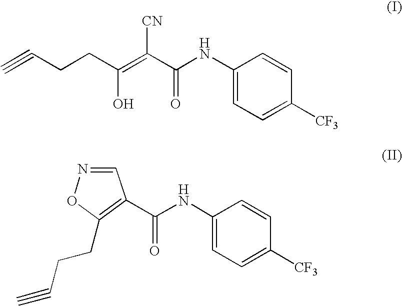
for the manufacture of a medicament for preventing and/or treating chronic rejection in a transplanted organ or tissue.

8. The use of claim 7 wherein the medicament is for preventing chronic rejection.

9. The use of claim 8 wherein the transplantation is allograft transplantation.

10. The use of claim 7 for the manufacture of the medicament with tacrolimus.

11. The use of claim 9 for the manufacture of the medicament with tacrolimus.

12. The use of claim 7 wherein the medicament is for oral administration.

13. A pharmaceutical composition for preventing and/or treating chronic rejection in a transplanted organ or tissue, which comprises a therapeutically effective amount of compound of the formula (I) or (II): 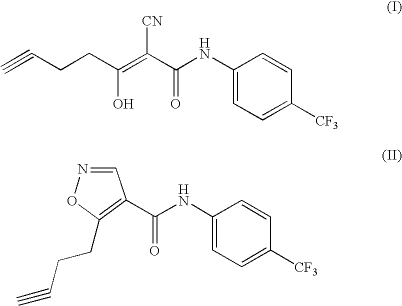
in admixture with a pharmaceutically acceptable carrier or excipient.

14. The pharmaceutical composition of claim 13 wherein the composition is for preventing chronic rejection.

15. The pharmaceutical composition of claim 14 wherein the transplantation is allograft transplantation.

16. The pharmaceutical composition of claim 13 which is for co-administering a therapeutically effective amount of tacrolimus.

17. The pharmaceutical composition of claim 15 which is for co-administering a therapeutically effective amount of tacrolimus.

18. The pharmaceutical composition of claim 13 which is for oral administration.


SEQUENCE LISTING (3 sequences)

SEQ ID NO: 1 - DNA, 19 bp, Artificial Sequence
  Synthetic DNA
gctgctgacccccactgat

SEQ ID NO: 2 - DNA, 18 bp, Artificial Sequence
  Synthetic DNA
gccactgccggacaactc

SEQ ID NO: 3 - DNA, 25 bp, Artificial Sequence
  Synthetic DNA
cgcctgagtggctgtcttttgacgt